(19) United States
(12) Patent Application Publication
(10) Pub. No.: US 2024/0093135 A1
(43) Pub. Date: Mar. 21, 2024

(54) BASE ELEMENT OF A MULTI-CHAMBER BIOCHIP, PRODUCTION OF THE MULTI-CHAMBER BIOCHIP, AND USE THEREOF FOR ESTABLISHING ORGAN AND DISEASE MODELS AND SUBSTANCE TESTS

(71) Applicant: Dynamic42 GmbH, Jena (DE)

(72) Inventors: Nader Abdo, Jena (DE); Knut Rennert, Jena (DE); Martin Raasch, Weimar (DE)

(21) Appl. No.: 18/469,439

(22) Filed: Sep. 18, 2023

(30) Foreign Application Priority Data
Sep. 18, 2022 (DE) 10 2022 123 877.6

(51) Int. Cl. C12M 1/32; C12M 1/00; C12M 3/00

(52) U.S. Cl. CPC C12M 23/12; C12M 21/08; C12M 23/34

(57) ABSTRACT

A base element of a multi-chamber biochip has a bottom with a frame situated thereon, open on its side facing away from the bottom, wherein an interior space is enclosed by the frame. Furthermore, at least one first web is present; however, preferably a first web and a second web are present. Culture chambers formed by the webs and the frame, and delimited from one another via membranes, are contacted via channels, and serve to generate biological model systems. The base element is in particular formed as a single piece (monolithically). A multi-chamber biochip, a set, and a method for making a multi-chamber biochip are also disclosed.

### CROSS-REFERENCE TO RELATED APPLICATIONS

This application claims priority of German patent application no. 10 2022 123 877.6, filed Sep. 18, 2022, the entire content of which is incorporated herein by reference.

### TECHNICAL FIELD

The disclosure relates to a base element which can be used to produce a multi-chamber biochip for a series of innovative applications.

### BACKGROUND

Biochips in the sense of this application serve to simulate biological systems, such as, for example, organs or tissues in an experimental setup, in that biological structures and situations/environmental conditions are recreated in reaction chambers artificially, but as close to reality as possible. For example, a biochip can be formed from a number of components arranged one above the other, cooperating to form at least one culture chamber, but more often two culture chambers. These can be separated from one another via membranes of selected properties, wherein the culture chambers are filled with one medium or several media. Optionally, various substances, such as, for example, nutrients, active substances, and synthetic materials, as well as aerosols, cells, microorganisms, and/or spheroids, can be introduced in a targeted manner into the media in order to simulate biological systems or specific situations/environments under controlled conditions.

One possibility for the configuration of such biochips is known, for example, from the publication by Raasch, M., et al. (Raasch, M., et al., 2016; An integrative microfluidically supported in vitro model of an endothelial barrier combined with cortical spheroids simulates effects of neuroinflammation in neocortex development; Biomicrofluidics 10; doi 10.1063/1.4955184). Two base elements, each forming a preliminary culture chamber, are combined with one separation membrane each, and subsequently joined together by adhesive bonding, then sealed with closure films and partly sealed with adhesives or connecting substances. Filling the three culture chambers, which are arranged one above the other and separated by the two separation membranes, takes place via channels likewise formed in the two base elements. In this way, at least two culture chambers can be created in a small space and can be operated and examined under laboratory conditions, that is, while monitoring, for example, the biological structures used and the composition, temperature, and, optionally, flow of the media. The use of such generic biochips in a cell culture incubator additionally enables operation in an ambient atmosphere with, for example, a controlled temperature and composition.

Detection of the processes in the interior of the culture chambers can take place, for example, via an optical analysis through the transparent windows of the biochip (for example, via transmitted light microscopy analyses, live cell imaging). Furthermore, substances can be introduced via the channels, through the separation membranes, in order to assess the barrier/seal/permeability of the biological structures or to evaluate transport processes on the biological structures, and/or to analyze the reactions of proteins on the surfaces and/or inside of the biological structures. Furthermore, biological structures and introduced substances, such as media (potentially enriched with the aforementioned substances) can be removed for optical, molecular biological, and chemical analyses.

However, a biochip as described above must be constructed in layers from a number of different elements, wherein each layer must be connected tightly to the respective adjacent elements; in addition, the requirements for the desired dimensions of the culture chambers and thus the determined flow and physiological conditions must be reliably met. This means that, in particular, the base elements have to be produced with high precision and dimensional accuracy, as a result of which the production costs are high.

In the experiments of the inventors, it was further found that the gluing of the base elements induces stresses which lead to a deformation of the biochip, and the adhesion is subject to aging processes which make the biochip leaky, losing its function. In addition, it is disadvantageous that the separation membrane must be introduced before the bonding of the base elements. This is associated with a risk of contamination of the membrane by, for example, adhesive.

### SUMMARY

An object of the disclosure is to propose an option which makes it possible to reduce or resolve the disadvantages known from the prior art, and to furnish a multi-chamber biochip which can be used for a plurality of experimental applications.

The object of the disclosure is achieved by a base element of a multi-chamber biochip which has a bottom with at least one frame which is located thereon and which is open at least on its side facing away from the bottom. In this case, an interior space is surrounded by the frame. The frame can be configured as a step that surrounds the interior space. In an option which is advantageous because it is easier to produce, this can also be created via a planar, and in particular rectangular, molding of the base element, which has an accordingly high material thickness, corresponding in particular to the height of the interior space. Several frames which delimit several interior spaces can be placed on the bottom. Alternatively, in the variant of the planar molding of the base element, several interior spaces can also be surrounded by the planar molding.

Furthermore, at least one first web which is seated on the bottom within each interior space, and which extends around a first surface, is formed, the height of which is less than the height of the frame. The first web can be configured in this case to be free-standing, or can be configured as a step in the frame. The web has a first lateral surface facing the interior space and a first support surface opposite the bottom. The first support surface is configured to support a first separation membrane. A lower preliminary culture chamber (first chamber) is surrounded by the first lateral surface of the first web and the surface surrounded by the first web. The remaining interior space between the first support surface and the upper side of the frame forms an upper preliminary culture chamber (second chamber). The surface surrounded by the first web can be polygonal, preferably square, and in particular rectangular, but also round. Advantageously, the support surface of the web extends around the enclosed area at a uniform height with respect to the bottom. The lower preliminary culture chamber and the upper preliminary culture chamber are each connected to the surrounding environment by at least one separate channel that opens at an outer side of the base element.

In further embodiments of the base element according to the disclosure, the lower preliminary culture chamber and/or the upper preliminary culture chamber are connected to the surrounding environment by at least two separate channels. In the assembled state of the multi-chamber biochip, the channels advantageously allow for a supply and/or discharge of media into/out of the culture chambers.

For example, to allow the cultivation of spheroids of approximately 1 mm size, at least one culture chamber and the associated channels are configured with a construction having a clear height or a diameter of more than 1 mm—for example, 1.1 or 1.2 mm.

The base element according to the disclosure is formed (monolithically) from a single workpiece, and preferably has the format of a microscopy slide (76 mm×26 mm±3 mm). The monolithic configuration allows easier handling compared to solutions according to the prior art, since it is not necessary to connect two or more base elements to one another, whose passages (channels, interior spaces, culture chambers, or the like) need to be aligned and glued before the connection. In addition, the base element is more stable in a monolithic construction; it retains its original shape (no stress-related bending of the body), is not leaky at adhesive points, and is therefore more practical in handling and use. In particular, higher flow rates of the supplied media (perfusion speeds) can be achieved without leaks occurring. This is advantageous in particular in the establishment of organ models, because perfusion speeds matching those in vivo must be maintained in such cases over the longest possible period of time. The resulting omission of glued points also advantageously has the consequence that the adhesives—which are absolutely necessary in the prior art, and which could interact disadvantageously with the biological structures—can be omitted. In addition, the requirements for dimensional accuracy of the base element according to the disclosure can be lower than for the biochip composed of several elements.

In a further advantageous embodiment of the base element, a second web is formed, which sits on the bottom within the interior space and extends around a second surface. Its height is less than the height of the frame, but greater than the height of the first web. The second web can also be configured to be free-standing, or can be configured as a step in the frame, and has a support surface lying opposite the bottom, hereafter referred to as the second support surface. The second support surface is configured to support a second separation membrane. Between the first support surface and the second support surface, the second web forms a second lateral surface facing the interior space. The second surface, surrounded by the second web, and the second lateral surface define a middle preliminary culture chamber (third chamber). The surface surrounded by the second web can be polygonal, preferably square, and in particular rectangular, but also round. Preferably, the geometric shape of the surface enclosing the second web corresponds to that of the surface surrounded by the first web. The middle preliminary culture chamber is also connected by at least one channel to an outer side of the base element.

As will be explained further below, a multi-chamber biochip with three or more culture chambers arranged one above the other can advantageously be produced via such an embodiment of the base element.

Advantageously, the material from which the base element is produced includes an injection-molded, biocompatible plastic. Examples of such plastics are polyesters such as polyurethanes (PU), polyimides, styrenes (SEBS), polypropylenes (PP), polystyrenes (PS), polycarbonate (PC), polyethylene terephthalate (PET), and cyclic polyolefins (COP and COC). In a manner corresponding to the material selection, the base element is produced as a single piece in an injection molding or casting process, using the corresponding injection molding or casting tools.

Biochips are often also produced from polydimethylsiloxanes (PDMS) in the prior art. However, the use of PDMS-based chips for complex cell, organ, and disease models is complicated, since, in contrast to the aforementioned plastics, the PDMS has to be activated in order to be suitable for biological structures such as cell cultures. In order to influence the systems of biological structures and media to be investigated via a biochip as little as possible, the materials of the biochip should ideally be inert. However, the frequently-used material PDMS is known to have high bond-forming capacity with some chemical compounds, for example (Auner et al., (2019): Chemical PDMS binding kinetics and implications for bioavailability in microfluidic devices; Lab Chip 19: 864-874). This bond-forming capacity can have a difficult-to-estimate influence on experiments (for example, substance tests) that are carried out with biochips made of PDMS. For example, substances with a log P greater than 1.8 and a low hydrogen donor count are adsorbed strongly on PDMS, which makes active substance testing and interpretation of the data obtained considerably more difficult. For example, the substance propiconazole has a log P of 3.72 and a hydrogen donor count of 0. It is very hydrophobic and can only be detected at less than 30% of the starting concentration in the culture medium after 24 hours in PDMS chips, since it binds irreversibly to PDMS (Auner et al. 2019). This problem relates primarily, but not exclusively, to the classic active substance group of small molecules. Many pharmaceutical active substances belong to this group of drugs, such that PDMS-based biochips are not suitable as test systems. In addition, a one-piece base element for a multi-chamber biochip made only of a single workpiece is not producible via PDMS, since, with this substance, the necessary separation membrane can be fixed only between two individual components.

In an advantageous embodiment of the base element according to the disclosure, it is therefore produced from polybutylene terephthalate (PBT).

PBT is a polymer which is commonly used to produce products that are subject to a high mechanical load and/or which repeatedly come into contact with hot media. Typical uses of PBT are, for example, plain bearings, valve parts, screws, parts for household appliances such as coffee machines or hair dryers, and parts for medical devices such as connectors for pulse oximeters, tips for electrosurgical instruments, and clips for breathing masks. The production of suitable PBT starting polymers can even be realized in a GMP-compliant manner. PBT is very suitable for injection molding due to its favorable cooling and process behavior.

This material, which is unusual for use in cell culture chambers, showed very low bond-forming capacity in tests, compared to a series of components of the media used, such that an influence of the material of the multi-chamber biochips, and in particular of the (preliminary) culture chambers, on the tests taking place in such a cell culture chamber can be advantageously reduced. For example, the inventors have found in tests for active substance adsorption with propiconazole and troglitazone that PBT is very suitable for active substance tests of substances up to a log P of 3.72 (log P propiconazole: 3.72; log P troglitazone: 3.60). After an incubation period of 24 h, at least 80% of the starting concentration of the propiconazole or the troglitazone is detectable in the culture medium.

In a further embodiment, the base element according to the disclosure can be configured such that portions of the channels are formed in the lateral surface, facing away from the frame, of the bottom and/or in the lateral surface, opposite the bottom, of the base element. Preferably, the channel portions in the lateral surface or in the lateral surfaces can be formed completely or partially as preliminary channel portions, due to the formation of circumferential channel boundary webs. These channel boundary webs are created in the base element, adjoin it, or project out of the base element. The channel boundary webs have inwardly-oriented lateral surfaces which bound a channel space and thus act as a channel wall. Support surfaces, which are provided for supporting a (closure) membrane or a channel cover, run on the upper side (end face) of the channel boundary webs.

The channels are each connected to connectors (for example, standard Luer format) for supplying or removing media. These connectors are advantageously formed opposite the bottom.

A window can be present in the bottom, for example, which allows the visual detection of processes in at least one of the culture chambers.

In order to obtain a multi-chamber biochip according to the disclosure, a base element according to the disclosure is provided. This serves as the base of the multi-chamber biochip and advantageously allows both efficient production and flexible adaptation to the respective requirements.

In the case of a fully-assembled and ready-to-use multi-chamber biochip, a first separation membrane is placed on and connected to the first support surface of the first web. A lower culture chamber is provided by the first web and the first separation membrane. Depending upon the embodiment of the base element, a second separation membrane is optionally placed on a second support surface of a second web, and connected to the web. In this case, a middle culture chamber is provided between the first separation membrane and the second separation membrane. A closure membrane is placed on the frame and is connected to the frame. This closure membrane delimits an upper culture chamber which, depending upon the configuration of the base element, is provided between the first separation membrane and the closure membrane or between the second separation membrane and the closure membrane.

Optionally, an additional closure membrane is provided, which sits on the lateral surface, facing away from the frame, of the bottom, and is connected thereto.

The separation membranes used are preferably films which, depending upon the material, thickness, and production thereof, can be flexibly integrated to prespecified degrees, and can be permeable as well as impermeable to gases, liquids, particles, and/or more complex molecules (semi-permeable). Preferred but non-exclusive materials for the separation membranes are polyethylene terephthalate or polycarbonate. The membranes preferably have pores with a size between 0.4 μm and 8 μm, and have a thickness between 5 and 50 μm, preferably 10 and 20 μm, and particularly preferably 12 μm. The separation membranes can be at least translucent, and preferably transparent, to at least one selected wavelength range, in order to enable improved optical detection of processes in at least one of the culture chambers. The closure membranes (sometimes also referred to as bonding films) can also preferably be integrated in a flexible manner, and can be selected to be correspondingly blocking or (semi-)permeable to certain classes of substance. The closure membranes can also be transparent for at least one selected wavelength range, in order to enable an optical detection of processes in at least one of the culture chambers. In some embodiments, the closure membranes can be configured as transparent closure films—for example, as polycarbonate films or polyethylene terephthalate films. Glass or polystyrenes or COC/COP are also possible materials from which the closure membranes can be made. The closure membranes (or closure films) can also function as a channel cover, since they extend over optionally existing channel boundary webs and rest on the support surfaces thereof, and are connected to them in a gas-tight and liquid-tight manner.

In a particular embodiment of the multi-chamber biochip, at least one of the separation membranes can have depressions, which are also referred to as (micro)cavities. Cells, cell composites, spheroids, and/or organoids can be colonized and/or cultured in these (micro)cavities.

An application example here is the improved/extended culture of spheroids and organoids over a period of up to 4 days compared to static cell culture.

In the configuration of the multi-chamber biochip with microcavities, spheroids and organoids can be cultured in an immobilized manner under (microfluidic) cell culture conditions with flow-through, without an additional embedding in (hydro)gels, which usually consist of proteins of the extracellular matrix in individual form or mixed forms. The microcavities can be between 500 and 1,500 μm in diameter, and preferably 800 μm. The gel-free culture allows better optical analysis during the culture period. In addition, the gel-free culture allows a gentler and non-destructive recovery of the intact cell tissue—in particular, spheroids and organoids from the biochip—for further analyses such as tissue sections, immunofluorescence stains, flow cytometry, ELISA-based assays, or tissue lysis for DNA/RNA analyses and Western Blot analyses.

The gel-free immobilized culture of spheroids and organoids enables further an easier co-culturing with blood vessel tissue arrangements (vascularization)—either on different separation membranes (indirect vascularization) or on the same (micro)cavity separation membrane or planar separation membrane (direct vascularization). In addition, it is possible for immobilized and vascularized spheroids and organoids with immune cells to be directly rinsed in the culture medium. The gel-free culture of spheroids and organoids considerably facilitates the migration of immune cells into the tissue of the spheroid and organoids.

The culture under flow-through and/or gel-free cell culture conditions allows better maintenance of the vitality of biological structures, due among other things to improved nutrient and oxygen conditions—in particular, for spheroids—than under comparable static cell culture conditions. This makes it possible to maintain the function of such biological structures for a longer time for test purposes.

In order to provide a user with a wide selection of possibilities of use, a set for a multi-chamber biochip according to the disclosure can be provided, which includes a base element as described above. Furthermore, at least one first separation membrane is present in such a set, which serves for placement on the first web and closing off the lower culture chamber. Optionally, at least one second separation membrane is included in the set, which serves for placement on the second web and closing off the middle culture chamber if the base element included in the set has a second web. In addition, a closure membrane is included in the set, which serves for placement on the frame and closing off the remaining interior space as an upper culture chamber.

In addition, an additional closure membrane can be included, which is intended for placement on the lateral surface, facing away from the frame, of the bottom.

A set according to the disclosure can, for example, be provided directly to a user or delivered to a service provider who performs the assembly of the components of the set on behalf of and according to the specifications of, for example, the user.

Advantageously, the provision of such a set also enables the introduction of biological material, for example, larger organoids or cell clusters or tissue pieces or multi-cellular organisms, for example, parasites, which, due to their size, cannot be flushed into the chambers by the channels present. Such material can be applied directly in a sterile environment into a still-open preliminary culture chamber, which is subsequently closed off with a separation membrane or a closure membrane—for example, via an adhesive method.

The multi-chamber biochip is produced by providing a base element, and by the first separation membrane being placed on the first web. This is connected to the first web, forming a seal. If necessary, a biological material can be introduced into the lower preliminary culture chamber before the first separation membrane is applied, as described further above.

In the sense of this description, a sealed connection is understood to mean that a planar or linear connection is created, which is in particular liquid- and gas-tight, so that a culture chamber bounded by the membrane or a channel bounded by the membrane reliably withstands the flow of a medium under a certain static or dynamic working pressure.

A connection is advantageously—but not exclusively—made by guiding a beam of directed and controlled high-energy radiation along the joining seam or connecting surface to be produced. The high-energy radiation is in particular laser radiation of a wavelength and intensity which are matched to the materials to be connected.

Once the first separation membrane is attached to the first web, the second separation membrane is optionally placed on the second web and connected thereto, forming a seal. Optionally, a biological material can again be introduced into the middle preliminary culture chamber before the second separation membrane is applied. In a corresponding manner, the closure membrane is placed on the frame, and the closure membrane and frame are connected, forming a seal. Optionally, the additional closure membrane is placed on the lateral surface, facing away from the frame, of the bottom, and is connected thereto, forming a seal.

Since each preliminary culture chamber, and, as a result, each of the resulting culture chambers, is contacted by at least one channel, a medium can be supplied and/or discharged into each of the culture chambers of the multi-chamber biochip.

The above-described plurality of specific embodiments of the multi-chamber biochip according to the disclosure advantageously allows a considerable number of possibilities for using such a multi-chamber biochip. All of the intended uses include at least the following steps. First, a multi-chamber biochip according to the disclosure is selected and provided. The selection can take place, for example, with regard to the existing number of culture chambers and/or with regard to the choice and/or the combination of the separation membrane(s). In order to avoid undesirable interactions and contamination during use, the multi-chamber biochip can subsequently be sterilized. An assembly of sterile components under elevated purity conditions is also equivalent to a sterilization. Subsequently, for example, media, cells, microorganisms, spheroids and/or organoids and/or cell clusters and/or tissue pieces, or multi-cellular organisms can be introduced into the existing culture chambers.

In a specific embodiment of a use of the multi-chamber biochip or in one of its provided configurations, a hydrogel—preferably consisting of components of the extracellular matrix, such as, for example, collagens, fibronectin, laminins, et cetera—can be introduced into at least one of the culture chambers.

The multi-chamber biochip according to the disclosure can be used, for example, to generate and/or culture spheroids and/or organoids in at least one of its culture chambers by colonizing them in the (micro)cavities of one of the membranes.

The multi-chamber biochip according to the disclosure can also be used for testing cells, cell cultures, organoids, or spheroids with various substances, active substances, nanomaterials, microorganisms, vectors, antibodies, et cetera.

Due to its structure on the basis of the base element according to the disclosure, the disclosure is easy to use for a user. The assembly of a multi-chamber biochip is considerably simplified and significantly less prone to error compared to the prior art. Moreover, due to the one-piece configuration of the base element, an efficient production and a versatile combinability with a wide variety of separation membranes and/or closure membranes is possible. The use, for example, of the method of laser bonding to connect the membranes, forming a seal, makes it possible to dispense with glues or other adhesives.

A multi-chamber biochip according to the disclosure can, for example, be used, depending upon the specific embodiment, for active substance tests or the establishment and characterization of organ or organoid models and disease and infection models. For active substance tests, it is conceivable, for example, to examine an immune response of the cultured cells to a substance administration. In this case, there are different possibilities; the substance can be put into the chamber in which the cells to be tested grow, for example. The influence of the substance on the cells can then be determined, for example, by microscopic observation of the cells or by examination of the cell culture medium—for example, for messenger substances, markers, et cetera, discharged from the cells into the medium. Alternatively, the substance can also be added to the chamber opposite to the cells to be tested in order, for example, to examine an influence of cells growing in this chamber, located opposite to the cells to be tested, upon the effect of the substance in the cells to be tested of the other chamber, which could, for example, weaken or potentiate a substance effect (gradient formation). Furthermore, spheroids and/or organoids with and without immune cell populations can be rinsed/perfused. The immune cells can be rinsed/perfused into the chamber with the spheroids or organoids, or can preferably be flushed/perfused via a blood vessel structure in one of the adjacent chambers. In addition, the immune cells can be permanently integrated into the blood vessel structure and/or the spheroid/organoids. In addition, spheroids or organoids can be vascularized by introducing blood vessel cells such as endothelial cells alone or endothelial cells in combination with—but not exclusively—pericytes and smooth muscle cells. In order, for example, to simulate a tissue traversed by blood vessels, the upper and lower walls of the middle culture chamber can be lined with endothelial cells alone or in combination with pericytes and smooth muscle cells, which are introduced through the inlet channel, whereas organ-specific epithelial cells are introduced into the lower and into the upper culture chambers. In a further embodiment, however, the endothelial cells can also be introduced on the upper and lower walls of the upper or lower chamber by the given inlet channel, alone or in combination with pericytes and smooth muscle cells, and, in the middle chamber, epithelial tissue can be integrated in the form of layered cell layers or in the form of spheroids and organoids.

### BRIEF DESCRIPTION OF DRAWINGS

The invention will now be described with reference to the drawings wherein:

FIG. 1 is a schematic perspectival view of an embodiment of a base element according to the disclosure;

FIG. 2 is a schematic view of an embodiment of channels in the bottom of a base element according to the disclosure;

FIG. 3 is a schematic view of an embodiment of a set according to the disclosure for providing a multi-chamber biochip (exploded view);

FIG. 4 is a lateral section through a multi-chamber biochip according to the disclosure, with three culture chambers, and a schematic illustration of an apparatus for operating the multi-chamber biochip; and,

FIG. 5 shows a possible use of a multi-chamber biochip according to the disclosure.

### DETAILED DESCRIPTION

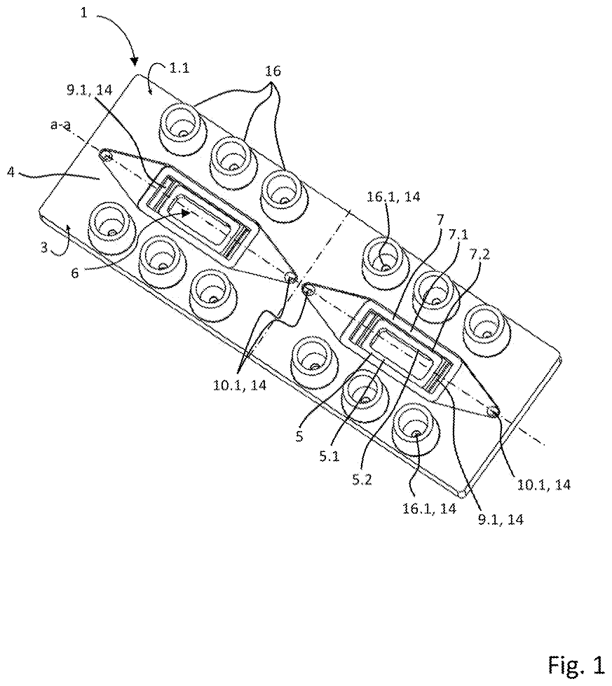

In the following, the disclosure is described by embodiments in which two interior spaces 6 are respectively bounded by a rectangular planar molding of a frame 4 on a bottom 3 of a base element 1. As will be explained in more detail below, the interior spaces 6 are divided in a stepped fashion in such a way that they each form a (preliminary) multi-chamber cavity 2.1. A multi-chamber biochip 2 can thus be provided by the base element 1 of FIGS. 1, 2, and 3, or a multi-chamber biochip 2 is provided, which has two multi-chamber cavities 2.1 (wherein both multi-chamber cavities 2.1 functionally form a complete multi-chamber biochip). To improve the clarity of the figures, both existing multi-chamber cavities 2.1 are used in FIGS. 1, 2, and 3 to indicate the elements of a single multi-chamber cavity 2.1 or of a multi-chamber biochip 2 with only one multi-chamber cavity 2.1. The description relates here only to one multi-chamber cavity 2.1.

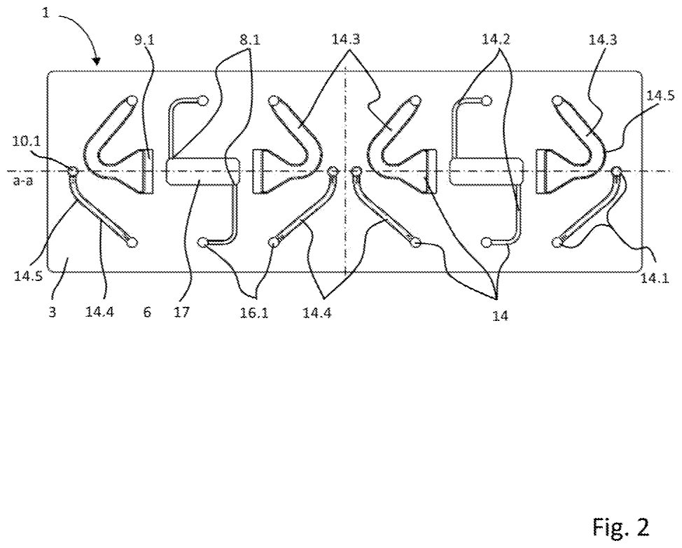

A base element 1 according to the disclosure is formed as a single piece from a biocompatible material, and in particular from a biocompatible injection-molded plastic (FIG. 1). Starting from a bottom 3 (see also FIG. 2), a frame 4 is formed, which is open on its side facing away from the bottom. The frame 4 is planar and encloses an interior space 6.

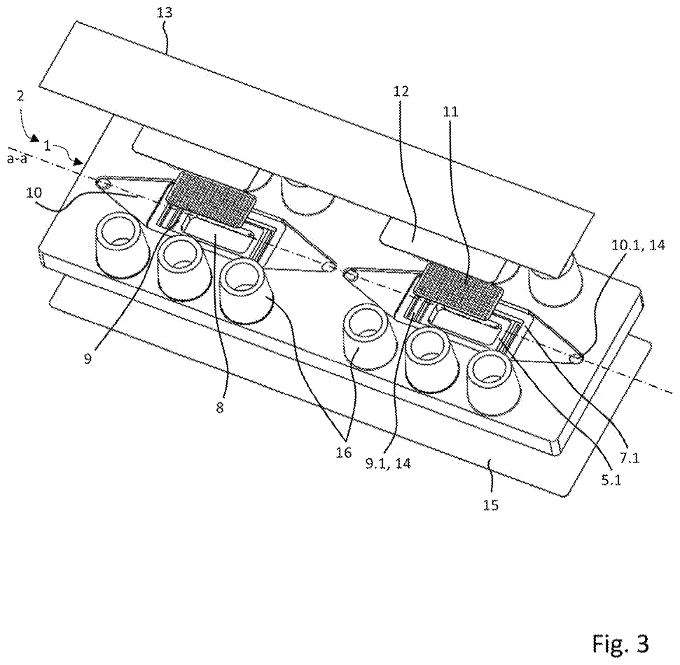

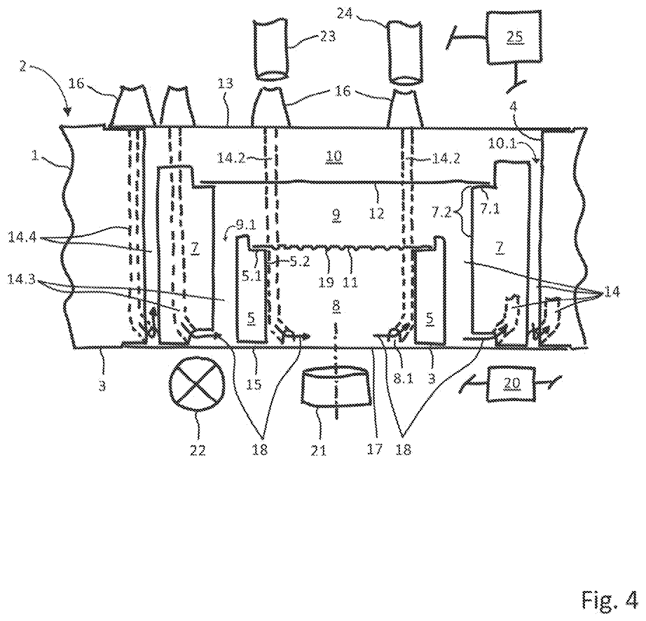

Within the interior space 6, a first web 5, which extends around a first surface, is made in the form of a step present in the material of the frame 4. The height of the first web 5 is less than the height of the frame 4. The first web 5 has a first lateral surface 5.2, facing the interior space 6, and a first support surface 5.1 opposite the bottom 3. The first support surface 5.1 is configured to support a first separation membrane 11 (see FIGS. 3, 4). A lower preliminary culture chamber 8 is bounded by the first lateral surface 5.2 and the surface enclosed by the first web 5. In the embodiment of FIGS. 1 through 3, the surface enclosed by the first web 5 is rectangular.

In addition, a second web 7 which is placed inside the interior space 6 on the bottom 3 and surrounds a second surface is provided, which is likewise configured as a step made of material of the base element 1 (FIG. 1). The second surface enclosed by the second web 7 is larger than the first surface enclosed by the first web 5, and also rectangular. The height of the second web 7 is less than the height of the frame 4, but greater than the height of the first web 5. A second support surface 7.1 is formed on the second web 7. The second support surface 7.1 is configured to support a second separation membrane 12 (see FIG. 3). In the portion between the first support surface 5.1 of the first web 5 and the second support surface 7.1 of the second web 7, the second web 7 forms a second lateral surface 7.2 facing the interior space 6. A middle preliminary culture chamber 9 is bounded by the second surface, surrounded by the second web 7, and the second lateral surface 7.2. The remaining interior space 6 between the second support surface 7.1 and an upper side 1.1 of the base element 1 forms an upper preliminary culture chamber 10. The upper side 1.1 is formed by the lateral surface, opposite the bottom 3, of the base element 1.

In order to be able to supply the culture chambers 8, 9, and 10 resulting from the preliminary culture chambers 8, 9, 10 in the assembled state of the multi-chamber biochip 2 with media 18 (see FIG. 4), two channels 14 are formed between the culture chambers 8, 9, and 10 and the upper side 1.1 of the base element 1. In FIG. 1, the channels 14 can be seen as round passages 10.1, 16.1 and as passages with rectangular cross-sections 9.1. The channels 14 lead from the culture chambers 8, 9, and 10 in the direction of the bottom 3, where a distribution to the connectors 16 takes place (cf. FIGS. 1, 3, 4).

In the embodiment shown, the connectors 16 sit on the upper side 1.1 of the base element 1. In the embodiment, each of the connectors 16 is configured for the supply and discharge of media 18 through each of the culture chambers 8, 9, 10. Each of the culture chambers 8, 9, 10 of a multi-chamber biochip 2 ready for operation can therefore allow passage of a medium 18 independently of the other culture chambers 8, 9, 10. In particular, each medium 18 can be selected individually for the respective culture chambers 8, 9, 10 and can be applied to the respective culture chambers 8, 9, 10 by an individually controllable volume flow.

To enable an optical detection of processes at least in the lower culture chamber 8 during the operation of the multi-chamber biochip 2, a window 17 is formed in the bottom 3 (FIG. 4). The surface of the window 17 is congruent with the surface enclosed by the first web 5.

The course of the channels 14 and the connection thereof to the respective connectors 16 is shown in FIG. 2 with a representation of the outwardly-pointing lateral surface of the bottom 3. For the supply and discharge of a medium 18 through the lower culture chamber 8, two lower channels 14.2 are present which open into the lower culture chamber 8 through two supply openings 8.1 arranged diagonally opposite one another. To supply the middle culture chamber 9 with a further medium 18, two middle channels 14.3 are provided, which are formed in portions as opposite rectangular supply passages 9.1 arranged between the first web 5 and the second web 7 (see also FIG. 1). The upper culture chamber 10 is supplied with a further medium 18 by two upper channels 14.4, in that the latter contact the upper culture chamber 10 via the respectively outermost, round supply passages 10.1.

The connector passages 16.1 shown in each case with a round cross-section then produce the respective connections to the connectors 16.

In the embodiment of FIG. 2, portions 14.1 of the channels 14 between the supply openings 8.1 and the supply passages 9.1, 10.1, on the one hand, are present, as are, on the other, the associated connector passages 16.1 which are fashioned as preliminary channel portions 14.1. The preliminary channel portions 14.1 are formed by depressions incorporated into the base element 1. In the embodiment, the depressions are arranged in the shape of a groove with a rectangular cross-section. Of course, other types of depressions are also possible with other cross-sections—such as a semicircular cross-section. The depressions have inner channel walls 14.5, each enclosing a connector passage 16.1 and a supply passage 9.1, 10.1, or, in the case of the lower culture chamber 8, the two associated connector passages 16.1 and the window 17 with the two, diagonally opposite supply passages 8.1. The channel walls 14.5 sit flush with the lateral surface, pointing away from the frame 4, of the bottom 3. By applying a channel cover, the preliminary channel portions 14.1 can be closed, and thus the functional state of these channel portions 14.1 can be produced. In the embodiment of FIGS. 3 and 4, a lower closure membrane 15 is applied to the lateral surface, pointing away from the frame 4, of the bottom 3, and, in the manner of a seam, along the channel walls 14.5 is connected in a liquid-tight manner to the bottom 3. The connection is preferably carried out by laser welding along the connection seam to be produced, but can also be effected, for example, by gluing or solvent welding. Due to the liquid-tight connection of the closure membrane 15, the preliminary channel portions 14.1 attain their functional state.

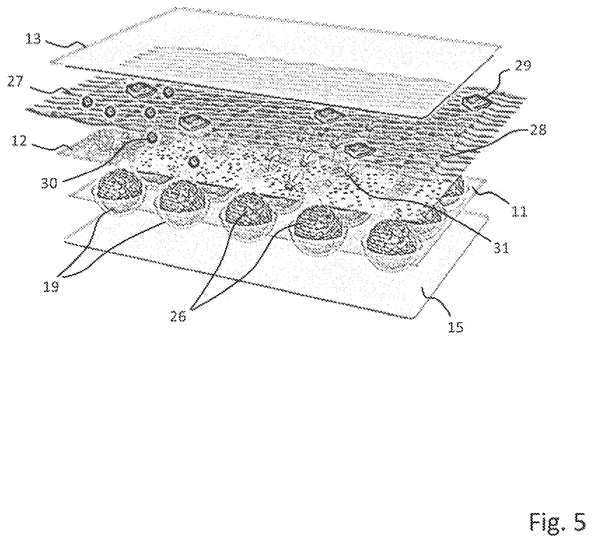

In FIG. 3, a set for a multi-chamber biochip 2 is shown by way of example, wherein the illustration can also be regarded as an exploded illustration of the components of an embodiment of a multi-chamber biochip 2. A base element 1 according to the disclosure is present as a central element. A first flexible separation membrane 11 is provided for placement on the first support surface 5.1 of the first web 5. If the first separation membrane 11 is attached there, it closes off a lower culture chamber 8. The first separation membrane 11 is provided, for example, with microcavities 19, into which, for example, spheroids or organoids can be introduced, and/or can be cultivated there (see FIG. 5). A second flexible separation membrane 12 serves for placement on the second support surface 7.1 of the second web 7, and closes off a middle culture chamber 9. A transparent closure membrane 13, which is also provided, can be placed on the frame 4, which membrane in the assembled state serves to close off the remaining interior space 6 as an upper culture chamber 10 between the second separation membrane 12 and the closure membrane 13.

In order also to close off the channel portions 14.1, formed in the bottom 3, as well as the window 17, an additional transparent closure membrane 15 is present, which, as already explained above, is applied on the bottom 3 and spans and seals the respective channel portions 14.1 and the window 17.

A multi-chamber biochip 2 according to the disclosure, with a lower culture chamber 8, a middle culture chamber 9, and an upper culture chamber 10, is shown in FIG. 4 in a lateral sectional view. The drawing plane of FIG. 4 corresponds to the line a-a of FIG. 3, wherein only the left half of the multi-chamber biochip of FIG. 3 is shown in FIG. 4 for reasons of clarity.

The monolithic structure of the base element 1 of the multi-chamber biochip 2 can be clearly seen in FIG. 4. Starting from a bottom 3, a flat frame 4 is formed which is open on its side facing away from the bottom 3. Within the frame 4, a first web 5, which extends around a first surface, is made in the form of a step present in the material of the frame 4. The height of the first web 5 is less than the height of the frame 4. The first web 5 has a first lateral surface 5.2 and a first support surface 5.1 opposite the bottom 3. In the region of the surface enclosing the first web 5, the bottom has a window 17. Furthermore, there is a second web 7 which is placed on the bottom 3 and surrounds a second surface, and is likewise configured as a step made of material of the base element 1. The height of the second web 7 is less than the height of the frame 4, but greater than the height of the first web 5. A second support surface 7.1 is formed on the second web 7. Between the first support surface 5.1 of the first web 5 and the second support surface 7.1 of the second web 7, the second web 7 forms a second lateral surface 7.2 facing the interior space 6.

A first separation membrane 11 is applied to the first support surface 5.1, and a second separation membrane 12 is applied to the second support surface 7.1—in both cases in a liquid-tight manner. The first separation membrane 11 has microcavities 19. On the lateral surface, facing away from the frame 4, of the bottom 3, a lower closure membrane 15 is applied, and an upper closure membrane 13 is applied to the upper side of the frame—in both cases in a liquid-tight manner. A lower culture chamber 8 is bounded by the first lateral surface 5.2, the first separation membrane 11, and the lower closure membrane 15. A middle culture chamber 9 is bounded by the second lateral surface 7.2, the first separation membrane 11, and the second separation membrane 12, and an upper culture chamber 10 is bounded by the remaining frame 4, the second separation membrane 12, and the upper closure membrane 13.

The functions of the membranes 11, 12, 13, and 15 are clearly visible—both to delimit the culture chambers 8, 9, and 10 from one another or from the surrounding environment, and to provide desired options with regard to the exchange of molecules and/or cells between the culture chambers 8, 9, and 10.

In the examples of FIGS. 3 and 4, the multi-chamber biochip 2 has three chambers. The lower chamber 8 has a usable base area of approximately 42 mm2, a height of 0.5 mm, and a volume of 60 mm3, including the volume of the inlet and discharge channels 14. In the region of the supply openings 8.1, the inlet and discharge channels 14 have a diameter of approximately 0.5 mm. The middle chamber 9 has a usable base area of 160 mm2, a height of 1.1 mm, and a volume of just 200 mm3, including the volume of the inlet and discharge channels 14. The inlet and discharge channels 14 are, in the region of the supply passages 9.1, rectangular and 2 mm wide. The upper chamber 10 has a usable base area of 216 mm2, a height of 0.7 mm, and a volume of around 150 mm3, including the volume of the inlet and discharge channels 14. The inlet and discharge channels 14 have a diameter of 0.8 mm in the region of the supply passages 10.1. All mentioned single-dimensional specifications can vary and, for example, have a variability of ±0.5 mm, which can result in changes in the size of the surfaces and volumes.

Various media 18 (shown by arrows) can flow along the associated channels 14 into the respective culture chambers 8, 9, 10 and out again, wherein the supply of media 18 into the culture chambers 8, 9, 10 can take place independently of one another (FIG. 4). The connection principle is shown in FIG. 4 using the example of the lower culture chamber 8: the associated connectors 16, via which a supply line 23 and a discharge line 24 are arranged in each case, are connected to lower channels 14.2, which open into the lower culture chamber 8 via supply openings 8.1 and supply the lower chamber 8 with a medium 18. The lower channels 14.2 extend in part offset to the drawing plane and are therefore drawn with partially dashed lines.

In an analogous manner, the middle culture chamber 9 can be supplied with medium 18 via the middle channels 14.3, and the upper culture chamber 10 via the upper channels 14.4. The multi-chamber biochip 2 can be operated, for example, via a device such as a reading device or a microscope which has a lens 21 which is oriented towards the window 17, and which can be monitored and optionally detected, stored, and evaluated via the processes in the multi-chamber biochip 2. For this purpose, a light source 22 can also be present in order to illuminate the multi-chamber biochip 2 in the desired manner. In addition, a pump 25 can be arranged which is connected to the supply lines 23 and discharge lines 24, which in turn are attached to the corresponding connectors 16. The pump 25 and optionally the light source 22 can be controlled via a controller 20 so that, for example, a perfusion of the culture chambers 8, 9, 10 can be carried out in a controlled manner and can be monitored optically. The controller 20, which is implemented, for example, by a computer, can optionally also store and/or evaluate optically-detected data—in addition to the generation of control commands. It is possible, for example, to control the pumping rates for the individual culture chambers 8, 9, 10 as a function of the optically-detected data via the controller 20.

The disclosure advantageously enables the construction of complex biological models. For example, microfluidic cultures of spheroids 26 and/or organoids 26 with integrated blood vessel and immune cell circulation can be realized.

Thus, a model for studying pancreatic cancer (PDAC, pancreatic ductal adenocarcinoma) can be created (FIG. 5). For this purpose, the lower culture chamber 8 is provided with a clear height of 0.5 mm between the lower closure membrane 15 and the first separation membrane 11. The first separation membrane 11 is porous and is provided with microcavities 19 on its lateral surface facing the middle culture chamber 9, in which cavities the spheroids 26 can be colonized and cultured. The middle culture chamber 9 has a clear height of 1.1 mm, so that spheroids 26 can be introduced in a non-destructive manner up to a size of approximately 1 mm. The channels 14 are also dimensioned to be correspondingly large.

In the embodiment of FIG. 5, the second separation membrane 12 is configured as a porous PET film and covers the middle culture chamber 9. A cell layer 27 consisting of microvascular, pancreatic endothelial cells 28 with macrophages 29 is formed in the upper culture chamber 10. For the sake of improved clarity, the cell layer 27 is shown at a distance from the second separation membrane 12. In reality, the cell layer 27 grows adherently on the second separation membrane 12. Through the second separation membrane 12, perfused monocytes 30 and T-cells 31, for example, can pass into the middle culture chamber 9.

The upper culture chamber 10 has a clear height of 0.7 mm between the second separation membrane 7 and the closure membrane 13.

It is understood that the foregoing description is that of the preferred embodiments of the invention and that various changes and modifications may be made thereto without departing from the spirit and scope of the invention as defined in the appended claims.

### LIST OF REFERENCE SIGNS

1 base element
1.1 upper side
2 multi-chamber biochip
2.1 multi-chamber cavity
3 bottom
4 frame
5 first web
5.1 first support surface (first end face)
5.2 first lateral surface
6 interior space
7 second web
7.1 second support surface (second end face)
7.2 second lateral surface
8 lower (preliminary) culture chamber
8.1 supply opening
9 middle (preliminary) culture chamber
9.1 supply passage
10 upper (preliminary) culture chamber
10.1 supply passage
11 first separation membrane
12 second separation membrane
13 upper closure membrane (or upper bonding film)
14 channel
14.1 preliminary channel portion
14.2 lower channel
14.3 middle channel
14.4 upper channel
14.5 channel wall
15 additional (lower) closure membrane (or lower bonding film)
16 connector
16.1 connector passage
17 window
18 medium
19 microcavity
20 controller
21 lens
22 light source
23 inlet
24 discharge
25 pump
26 spheroid/organoid
27 cell layer
28 (pancreatic) endothelial cell
29 macrophages
30 monocytes
31 T-cells

1. A base element of a multi-chamber biochip formed from a single workpiece, the base element comprising:
a bottom having a frame disposed thereon;
said frame having an elevation and having a side facing away from said bottom and being open on said side facing away from said bottom;
said frame delimiting an interior space;
a first web seated inside said interior space on said bottom and surrounding a first surface;
said first web defining a first support surface and a first lateral surface having an elevation less than said elevation of said frame;
a lower preliminary culture chamber conjointly enclosed by said first surface surrounded by said first web and said first lateral surface;
said frame having an upper side;
an upper preliminary culture chamber being formed between said upper side and said first support surface;
a first channel opening on an outer side of said base element and configured to connect said lower preliminary culture chamber to an ambient; and,
a second channel opening on an outer side of said base element and configured to connect said upper preliminary culture chamber to the ambient.

2. The base element of claim 1, further comprising:
a second web extending within said interior space around a second surface;
said second web having an elevation less than said elevation of said frame and greater than said elevation of said first web;
said second web having a second lateral surface;
a middle preliminary culture chamber enclosed by said second lateral surface of said second web and said surface enclosing said second web; and,
a third channel opening on an outer side of said base element and being configured to connect said middle preliminary culture chamber to the ambient.

3. The base element of claim 1, wherein said base element is made from a material comprising an injection-molded, biocompatible plastic.

4. The base element of claim 3, wherein said biocompatible plastic is polybutylene terephthalate (PBT).

5. The base element of claim 1, wherein said bottom has a lateral surface facing away from said frame; said channels have segments formed in said lateral surface of said bottom; and, said base element further comprises connectors connected to corresponding ones of said channels to supply or discharge media.

6. The base element of claim 5, wherein the segments of said channels are formed completely or partially as preliminary channel segments.

7. The base element of claim 1, wherein said frame is a first frame and said base element further comprises a second frame having a first web.

8. The base element of claim 7, wherein a second web is formed on said bottom.

9. A multi-chamber biochip comprising:
a base element formed from a single workpiece and including:
a bottom having a frame disposed thereon;
said frame having an elevation and having a side facing away from said bottom and being open on said side facing away from said bottom;
said frame delimiting an interior space;
a first web seated inside said interior space on said bottom and surrounding a first surface;
said first web defining a first support surface and a first lateral surface having an elevation less than said elevation of said frame;
a lower preliminary culture chamber conjointly enclosed by said first surface surrounded by said first web and said first lateral surface;
said frame having an upper side;
an upper preliminary culture chamber being formed between said upper side and said first support surface;
a first channel opening on an outer side of said base element and configured to connect said lower preliminary culture chamber to an ambient;
a second channel opening on an outer side of said base element and configured to connect said upper preliminary culture chamber to the ambient;
a second web extending within said interior space around a second surface;
said second web having an elevation less than said elevation of said frame and greater than said elevation of said first web;
said second web having a second lateral surface;
a middle preliminary culture chamber enclosed by said second lateral surface of said second web and said surface enclosing said second web;
a third channel opening on an outer side of said base element and being configured to connect said middle preliminary culture chamber to the ambient;
said multi-chamber biochip further including:
a first separation membrane placed on said first web and connected thereto;
a lower culture chamber being provided by said first web and said first separation membrane;
a second separation membrane optionally placed on said second web and connected thereto;
a middle culture chamber being provided between said first separation membrane and said second separation membrane;
an upper closure membrane placed on said upper side of said frame and being connected thereto; and,
an upper culture chamber being provided in accordance with one of the following:
a) between said upper closure membrane and said first separation membrane; or,
b) between said upper closure membrane and said second separation membrane.

10. The multi-chamber biochip of claim 9, wherein said bottom has a lateral surface facing away from said frame; and, said multi-chamber biochip further comprises a lower closure membrane on said lateral surface of said bottom and is connected thereto.

11. The multi-chamber biochip of claim 9, wherein at least one of said separation membranes has microcavities.

12. A method for making a multi-chamber biochip, the multi-chamber biochip comprising: a base element formed from a single workpiece and including: a bottom having a frame disposed thereon; said frame having an elevation and having a side facing away from said bottom and being open on said side facing away from said bottom; said frame delimiting an interior space; a first web seated inside said interior space on said bottom and surrounding a first surface; said first web defining a first support surface and a first lateral surface having an elevation less than said elevation of said frame; a lower preliminary culture chamber conjointly enclosed by said first surface surrounded by said first web and said first lateral surface; said frame having an upper side; an upper preliminary culture chamber being formed between said upper side and said first support surface; a first channel opening on an outer side of said base element and configured to connect said lower preliminary culture chamber to an ambient; a second channel opening on an outer side of said base element and configured to connect said upper preliminary culture chamber to the ambient; a second web extending within said interior space around a second surface; said second web having an elevation less than said elevation of said frame and greater than said elevation of said first web; said second web having a second lateral surface; a middle preliminary culture chamber enclosed by said second lateral surface of said second web and said surface enclosing said second web; a third channel opening on an outer side of said base element and being configured to connect said middle preliminary culture chamber to the ambient; said multi-chamber biochip further including: a first separation membrane placed on said first web and connected thereto; a lower culture chamber being provided by said first web and said first separation membrane; a second separation membrane optionally placed on said second web and connected thereto; a middle culture chamber being provided between said first separation membrane and said second separation membrane; an upper closure membrane placed on said upper side of said frame and being connected thereto; and, an upper culture chamber being provided in accordance with one of the following: a) between said upper closure membrane and said first separation membrane; or, b) between said upper closure membrane and said second separation membrane; said bottom having a lateral surface facing away from said frame; and, said multi-chamber biochip further comprising a lower closure membrane on said lateral surface of said bottom and is connected thereto, the method comprising the steps of:
providing the base element;
placing the first separation membrane onto the first web and connecting the first separation membrane and the first web, forming a seal;
optionally placing the second separation membrane onto the second web and connecting the second separation membrane and the second web, forming a seal;
placing the upper closure membrane onto the frame and connecting the upper closure membrane and frame, forming a seal;
optionally placing the lower closure membrane onto the lateral surface, facing away from the frame, of the bottom, and connecting the bottom and the lower closure membrane, forming a seal.

13. The method of claim 12, wherein the sealed connection is effected by a directed and controlled bundle of high-energy radiation.

14. A set for a multi-chamber biochip of claim 9, the set comprising:
said base element;
at least one first separation membrane which serves for placement onto the first web and closing off the lower culture chamber;
optionally, at least one second separation membrane which serves for placement onto the second web and closing off the middle culture chamber; and,
an upper closure membrane which serves for placement onto the frame and closing off the upper culture chamber.

15. The set of claim 14, wherein an additional closure membrane which is intended for placement on the lateral surface, facing away from the frame, of the bottom.

16. A method for culturing cells, comprising the steps of:
selecting and providing a multi-chamber biochip according to claim 9; and,
introducing media and cells into the existing culture chambers.

17. The method of claim 16, wherein a hydrogel is introduced into at least one of the culture chambers.

18. The method of claim 16, wherein spheroids and/or organoids are produced and/or cultured in at least one of the culture chambers, by colonizing them in microcavities of at least one of the separation membranes.
